(19) United States
(12) Patent Application Publication
(10) Pub. No.: US 2013/0253020 A1
(43) Pub. Date: Sep. 26, 2013

(54) Treatment of Degenerative Disc Diseases

(71) Applicants: Tang Yue-Cheong Tang, St Louis, MO (US); Alok Sharan, New York, NY (US)

(72) Inventors: Tang Yue-Cheong Tang, St Louis, MO (US); Alok Sharan, New York, NY (US)

(21) Appl. No.: 13/749,614

(22) Filed: Jan. 24, 2013

Related U.S. Application Data
(60) Provisional application No. 61/590,307, filed on Jan. 24, 2012.

(51) Int. Cl. A61K 31/426

(52) U.S. Cl. CPC A61K 31/426
USPC 514/365

(57) ABSTRACT

The present disclosure provides methods treat or prevent a degenerative disc disease in a patient by administering to the patient a therapeutically effective amount of an advanced glycation endproduct (AGE) breaker. Methods for inhibiting the progression of a degenerative disc disease and for reducing the amount of an advanced glycation endproduct (AGE) in an intervertebral disc (IVD) are also provided.

### CROSS-REFERENCE TO RELATED APPLICATION

This application claims priority benefit under 35 U.S.C. §119 (e) of U.S. Provisional Application Ser. No. 61/590,307, filed Jan. 24, 2012, the content of which is hereby incorporated by reference in its entirety.

### FIELD OF THE DISCLOSURE

The present disclosure relates to the treatment of degenerative disc disease. More particularly, the present disclosure relates to the treatment of degenerative disc disease using a therapeutic agent or combination of agents that can modify and alleviate the disease state at the tissue and cellular level.

### BACKGROUND

Intervertebral Disc (“IVD”) degeneration is one of the strongest predictors of lower back pain, and it underlies several painful low back disorders including intervertebral disc herniation, degenerative spondylolisthesis, spinal stenosis, and degenerative disc disease (“DDD”).

With degeneration, the IVD matrix undergoes a cascade of changes including alterations of the collagen network, loss of proteoglycans, and decreased tissue hydration that compromise its load-bearing capacity. In addition, the IVD cells are responsible for matrix homeostasis shift towards catabolism with the elevated expression of proteases (MMPs & ADAMTs) leading to matrix degradation and the eventual collapse of the IVD structure. There are also increased vascular and nerve invasion into the disc structure. Furthermore, the IVD cells secrete pro-inflammatory cytokines (including TNF-α, IL-1β, -6, & -8) that may contribute to the symptomatic pain.

Although there are a number of causes for disc degeneration, disc degeneration has been associated with the accumulation of advanced glycation end-products (“AGEs”). The presence of AGEs reduces the ability of spinal discs to retain water. Dehydration and molecular crosslinking cause the spine to stiffen and become less flexible. On a cellular level, AGEs cause the increased production of cytokines relating to pain.

AGEs upregulate the cellular receptor of AGEs (RAGE), and the increased RAGE ligand expression and activation are linked with inflammatory pathways such as NFκB and TNFα signaling. RAGE and its isoforms sit in a pivotal role, regulating metabolism, inflammation, and epithelial survival in the setting of stress.

Current known treatments for severe disc degeneration involve invasive surgeries such as discectomies, spine fusions, and disc replacements. There are currently no effective treatments or therapies for early onset disc degeneration. The need and ability to treat early disc degeneration may reduce the progression to late stages of degeneration and alleviate the need for such heavily invasive procedures.

### SUMMARY

The present disclosure, in one embodiment, provides a method for treating or preventing a degenerative disc disease in a patient in need thereof. Also provided, in one embodiment, is a method for inhibiting the progression of a degenerative disc disease in a patient in need thereof. In one embodiment, provided is a method for reducing the amount of an advanced glycation endproduct (AGE) in an intervertebral disc (IVD) in a patient that suffers from an IVD degeneration disease.

The method, in some embodiments, entails administering to the patient a therapeutically effective amount of an advanced glycation endproduct (AGE) breaker or inhibitor.

Non-limiting examples of AGE inhibitors include ureido, aryl carboxamido phenoxyisobutyric acids and an N-phenacyl-thiazolium, pharmaceutically acceptable salts thereof, or biologically active metabolites thereof.

In one embodiment, the N-phenacyl-thiazolium is a compound of Formula I,

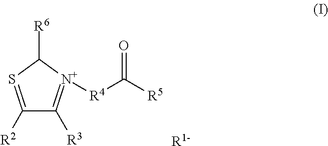

wherein:

R¹ is halo;

R² and R³ are each independently selected from H, alkyl or substituted alkyl;

R⁴ is alkyl or substituted alkyl;

R⁵ is phenyl or substituted phenyl; and

R⁶ is H, alkyl or substituted alkyl.

In another embodiment, the N-phenacyl-thiazolium is compound of Formula II,

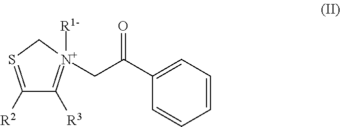

wherein:

R¹ is halo; and

R² and R³ are each independently selected from H, alkyl or substituted alkyl.

In some aspects, the patient is a mammal patient. In one aspect, the patient is a human patient.

Pharmaceutical compositions suitable for the disclosed methods are also provided.

### BRIEF DESCRIPTION OF THE DRAWINGS

Provided as embodiments of this disclosure are drawings which illustrate by exemplification only, and not limitation, wherein:

FIG. 1A shows the elastic changes corresponding to the increases of AGEs in annulus fibrosus (AF) and nucleus pulposus (NP) tissues;

FIG. 1B shows the viscoelastic changes corresponding to the increases of AGEs in annulus fibrosus (AF) and nucleus pulposus (NP) tissues;

FIG. 2 shows the rescuing effect of cleavage of the advanced glycation end-products using a solution containing N-phenacyl thiazolium bromide in the nucleus pulposus (NP) tissues; and

FIG. 3 shows the effect of N-phenacyl thiazolium bromide administration in reducing inflammatory cytokines in primary spinal disc cells. Interleukin-1 and -6 expression was observed to decrease with PTB administration. These effects are confirmed by the reduction in the expression of the Receptor-for-Advanced Glycation End-Products.

### DETAILED DESCRIPTION OF THE DISCLOSURE

Before the present compositions and methods are described, it is to be understood that the disclosure is not limited to the particular methodologies, protocols, cell lines, assays, and reagents described, as these may vary. It is also to be understood that the terminology used herein is intended to describe particular embodiments of the present disclosure, and is in no way intended to limit the scope of the present disclosure as set forth in the appended claims.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The practice of the present disclosure will employ, unless otherwise indicated, conventional methods of chemistry, biochemistry, molecular biology, cell biology, genetics, immunology, and pharmacology, within the skill of the art. Such techniques are explained fully in the literature. (See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; D. M. Weir, and C. C. Blackwell, eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press: Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton & Graham eds. (1997) PCR (Introduction to Biotechniques Series), 2nd ed., Springer Verlag).

### I. Methods

It is discovered that degenerative disc diseases are associated with accumulation of advanced glycation end-products (AGEs). Increased AGEs affect the ability of intervertebral disc (IVD) tissue to retain water and in turn directly affect the mechanical behavior of the tissue. Furthermore, the loss of water content was detectable by Magnetic Resonance Imaging (“MRI”) in a dose-dependent manner with the tissue concentration of AGEs. In addition, it is discovered that cytokines relating to pain were directly mediated by the presence of AGEs.

The present disclosure also demonstrates that reduction in the level of AGEs restored the tissue functional mechanical behavior comparable to a lesser degree of degeneration. Reduction of AGE levels, as the Examples show, can be achieved by administration of AGE breakers or inhibitors.

The post-translational modification known as non-enzymatic glycation can form AGEs, which occurs through the Amadori rearrangement of sugars and the amino residues on proteins. Long-lived structural proteins such as collagen and aggrecan, particularly in tissues with low turnover, such as the IVD, are susceptible to accumulation of AGEs. The changes induced by increased AGEs include specific deteriorations in the elastic and viscoelastic mechanical behaviors of the IVD tissues.

Investigations have been conducted on disc samples from human cadavers that had undergone in vivo accumulation and in vitro induction of the modified protein, resulting in disc degeneration. Exposure of the degenerated discs to the pharmacological compounds clearly demonstrated a significant reduction in the concentration of the modified protein and a corresponding restoration of the mechanical properties of the IVD tissue compared to untreated discs.

This disclosure therefore provides a method to reverse the adverse effects of AGEs in the degeneration of the IVD tissue through the localized delivery of AGEs cleaving compounds into the intervertebral disc. The directed administration of AGE breakers or inhibitors may alleviate the progression of degenerative disc disease. Pharmaceutical compounds such as N-Phenacyl-Thiazolium Bromide (PTB) that can specifically cleave AGEs have been identified as examples and are effective at reducing AGEs in proteins. It is therefore possible to identify early onset disc degeneration and allow the timely administration of AGE breakers to arrest the pathological progression of the disease.

The AGE breakers can be used for early treatment of disc degenerative disease through restoration of native IVD mechanical properties, and to treat the IVD tissue stiffening associated with scoliosis.

Therefore, the present disclosure unveils a new mechanism underlying the onset and progression of degenerative disc diseases, and demonstrates a new therapeutic method to inhibit or even reverse such progression.

In one embodiment, therefore, the present disclosure provides a method for treating or preventing a degenerative disc disease in a patient in need thereof, comprising administering to the patient a therapeutically effective amount of an advanced glycation endproduct (AGE) breaker.

Also provided, in another embodiment, is a method for inhibiting the progression of a degenerative disc disease in a patient in need thereof, comprising administering to the patient a therapeutically effective amount of an advanced glycation endproduct (AGE) breaker or inhibitor.

Yet in another embodiment, provided is a method for reducing the amount of an advanced glycation endproduct (AGE) in an intervertebral disc (IVD) in a patient that suffers from an IVD degeneration disease, comprising administering to the patient a therapeutically effective amount of an advanced glycation endproduct (AGE) breaker.

In some aspects, the degenerative disc disease comprises an intervertebral disc (IVD) degeneration disease. In some aspects, degenerative disc diseases also increases susceptibility or directly leads to other spinal complications including intervertebral disc herniation, degenerative spondylolisthesis, and spinal stenosis.

### II. Advanced Glycation Endproduct (AGE) Breakers

Various advanced glycation endproduct (AGE) breakers are known in the art, and have been shown to be able to inhibit or break down the glycation of amino acids, and thus reduce the amount of glycation as well as cross-linking of these amino acids.

Non-limiting examples of AGE inhibitors include ureido, aryl carboxamido phenoxyisobutyric acids and an N-phenacyl-thiazolium, pharmaceutically acceptable salts thereof, or biologically active metabolites thereof.

N-phenacylthiazolum bromide (“PTB”) is a known AGE breaker and is an N-phenacyl-thiazolium and has been used for this proof of concept. Other AGE breakers or inhibitors include ureido and aryl carboxamido phenoxyisobutyric acids (RAHBAR S, Figarola J L. Novel breakers of advanced glycation endproducts. Arch Biochem Biophys 2003; 419:63-79).

In one embodiment, the N-phenacyl-thiazolium is a compound of Formula I.

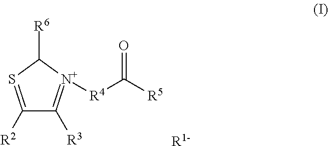

wherein:

R¹ is halo;

R² and R³ are each independently selected from H, alkyl or substituted alkyl;

R⁴ is alkyl or substituted alkyl;

R⁵ is phenyl or substituted phenyl; and

R⁶ is H, alkyl or substituted alkyl.

In another embodiment, the N-phenacyl-thiazolium is a compound of Formula II,

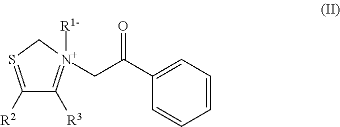

wherein:

R¹ is halo; and

R² and R³ are each independently selected from H, alkyl or substituted alkyl.

In one aspect, R¹ is Br or Cl. In another aspect, R¹ is Br.

In one aspect, R² and R³ are each independently selected from H or C₁-C₁₀ alkyl. In one aspect, R² and R³ are each independently selected from H or C₁-C₅ alkyl. In one aspect, R² and R³ are each independently selected from H, methyl or ethyl.

In one aspect, R⁴ is alkyl. In one aspect, R⁴ is C₁-C₅ alkyl. In one aspect, R⁴ is methyl or ethyl.

In one aspect. R⁵ is phenyl or phenyl substituted with one or more C₁-C₅ alkyl or one or more halo or one or more hydroxyl. In one aspect, R⁵ is phenyl or phenyl substituted with one or more methyl or ethyl. In one aspect, R⁵ is phenyl.

In one aspect, R⁶ is H, alkyl, such as C₁-C₅ alkyl. In one aspect, R⁶ is H, methyl or ethyl.

In some aspects, the AGE breaker is one or more selected from N-phenacyl thiazolium bromide (N-PTB), N-phenacyl-4,5-dimethylthiazolium bromide(PMTB), dimethyl-3-phenacylthiazolium chloride, L-bis-[4-(4-chorobenzamidophenoxy isobutryl) cystin (LR20); 4-(3,5-dichlorophenylureido)-phenoxyisobutyryl-1-amidocylohexane-1-carboxylic acid (LR23); methylene bis[4,4′-(2-chlorophenylureidophenoxyisobutyric acid)](LR90); 1,1-dimethylbiguanide (metformin); or 5-aminosalicylic acid (5-ASA), or pharmaceutically acceptable salts or biologically active metabolites. For example, but not limited to, those listed below:

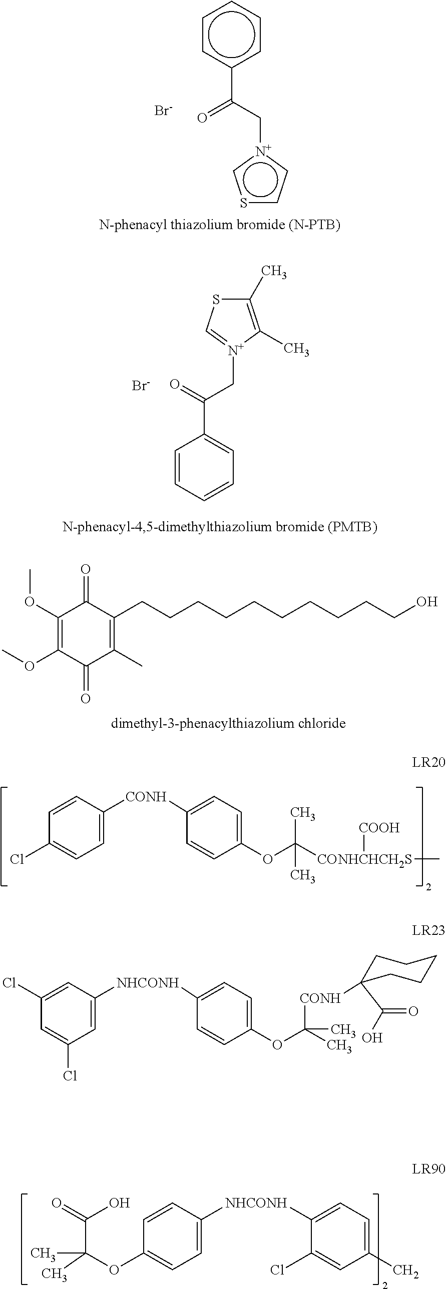

The term “alkyl” refers to saturated monovalent straight or branched chain hydrocarbyl groups having from 1 to 10 carbon atoms, more particularly from 1 to 5 carbon atoms, and even more particularly 1 to 3 carbon atoms. This term is exemplified by groups such as methyl, ethyl, n-propyl, iso-propyl, n-butyl, t-butyl, n-pentyl, and the like.

The term “substituted alkyl” refers to an alkyl group of from 1 to 10 carbon atoms, more particularly 1 to 5 carbon atoms, having from 1 to 5 substituents, preferably 1 to 3 substituents, independently selected from the group consisting of alkoxy, substituted alkoxy, acyl, acylamino, acyloxy, amino, substituted amino, aminoacyl, aminocarbonylamino, aminothiocarbonylamino, aminocarbonyloxy, aryl, substituted aryl, aryloxy, substituted aryloxy, aryloxyaryl, substituted aryloxyaryl, cyano, halogen, hydroxyl, nitro, oxo, thioxo, carboxyl, carboxyl esters, cycloalkyl, substituted cycloalkyl, thiol, alkylthio, substituted alkylthio, arylthio, substituted arylthio, cycloalkylthio, substituted cycloalkylthio, heteroarylthio, substituted heteroarylthio, heterocyclicthio, substituted heterocyclicthio, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, SO₃H, —S(O)ₙ-alkyl, —S(O)ₙ-substituted alkyl, —S(O)-aryl, —S(O)ₙ-substituted aryl, —S(O)ₙ-heteroaryl, —S(O)ₙ-substituted heteroaryl, —S(O)ₙ-cycloalkyl, —S(O)ₙ-substituted cycloalkyl, —S(O)-heterocyclic, —S(O)ₙ-substituted heterocyclic, where n is from zero to two, —OS(O)₂-alkyl, —OS(O)₂-substituted alkyl, —OS(O)₂-aryl, —OS(O)₂-substituted aryl, OS(O)₂-heteroaryl, —OS(O)₂-substituted heteroaryl, —OS(O)₂-heterocyclic, —OS(O)₂-substituted heterocyclic, and —OSO₂—NR⁴⁰R⁴⁰, —NR⁴⁰S(O)₂—NR⁴⁰-alkyl, —NR⁴⁰S(O)₂—NR⁴⁰-substituted alkyl, —NR⁴⁰S(O)₂—NR⁴⁰-aryl, —NR⁴⁰S(O)₂—NR⁴⁰-substituted aryl, —NR⁴⁰S(O)₂—NR⁴⁰-heteroaryl, —NR⁴⁰S(O)₂—NR⁴⁰-substituted heteroaryl, —NR⁴⁰S(O)₂—NR⁴⁰-heterocyclic, and —NR⁴⁰S(O)₂—NR⁴⁰-substituted heterocyclic, where each R⁴⁰ is independently selected from hydrogen or alkyl. This group is exemplified by groups such as benzyl, benzo[1,3]-dioxol-5-ylmethyl, etc.

The term “alkoxy” refers to the group “alkyl-O—” which includes, by way of example, methoxy, ethoxy, n-propoxy, iso-propoxy, n-butoxy, t-butoxy, sec-butoxy, n-pentoxy, and the like.

The term “substituted alkoxy” refers to the group “substituted alkyl-O—”.

The term “acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— provided that a nitrogen atom of the heterocyclic or substituted heterocyclic is not bound to the —C(O)— group wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “aminoacyl” and the prefix “carbamoyl” or “carboxamide” or “substituted carbamoyl” or “substituted carboxamide” refers to the group —C(O)NR⁴²R⁴² where each R⁴² is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or where each R⁴² is joined to form together with the nitrogen atom a heterocyclic or substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “alkenyl” refers to a vinyl unsaturated monovalent hydrocarbyl group having from 2 to 6 carbon atoms, and preferably 2 to 4 carbon atoms, and having at least 1, and preferably from 1 to 2 sites of vinyl (>C═C<) unsaturation. Such groups are exemplified by vinyl (ethen-1-yl), allyl, but-3-enyl and the like.

The term “substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic. This term includes both E (cis) and Z (trans) isomers as appropriate. It also includes mixtures of both E and Z components. It is understood that any hydroxyl substitution is not pendent to a vinyl carbon atom.

The term “alkynyl” refers to an acetylinic unsaturated monovalent hydrocarbyl groups having from 2 to 6 carbon atoms, and preferably 2 to 3 carbon atoms, and having at least 1, and preferably from 1 to 2 sites of acetylenic (—C≡C—) unsaturation. This group is exemplified by ethyn-1-yl, propyn-1-yl, propyn-2-yl, and the like.

The term “substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic. It is understood that any hydroxyl substitution is not pendent to a vinyl carbon atom.

The term “amino” refers to the group —NH₂.

The term “substituted amino” refers to the group —NR⁴¹R⁴¹, where each R⁴⁰ is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, and —SO₂-substituted heterocyclic, provided that both R⁴¹ groups are not hydrogen; or the R⁴¹ groups can be joined together with the nitrogen atom to form a heterocyclic or substituted heterocyclic ring. This group is exemplified by phenylamino, methylphenylamino, and the like.

The term “acylamino” refers to the groups —NR⁴⁵C(O)alkyl, —NR⁴⁵C(O)substituted alkyl, —NR⁴⁵C(O)cycloalkyl, —NR⁴⁵C(O)substituted cycloalkyl, —NR⁴⁵C(O)alkenyl, —NR⁴⁵C(O)substituted alkenyl, —NR⁴⁵S(O)alkynyl, —NR⁴⁵C(O)substituted alkynyl, —NR⁴⁵C(O)aryl, —NR⁴⁵C(O)substituted aryl, —NR⁴⁵C(O)heteroaryl, —NR⁴⁵C(O)substituted heteroaryl, —NR⁴⁵C(O)heterocyclic, and —NR⁴⁵C(O)substituted heterocyclic where R⁴⁵ is hydrogen or alkyl, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are defined herein.

The term “oxycarbonylamino” refers to the groups —NR⁴⁶C(O)O-alkyl, —NR⁴⁶C(O)O-substituted alkyl, —NR⁴⁶C(O)O-alkenyl, —NR⁴⁶C(O)O-substituted alkenyl, —NR⁴⁶C(O)O-alkynyl, —NR⁴⁶C(O)O-substituted alkynyl, —NR⁴⁶C(O)O-cycloalkyl, —NR⁴⁶C(O)O-substituted cycloalkyl, —NR⁴⁶C(O)O-aryl, —NR C(O)O-substituted aryl, —NR⁴⁶C(O)O-heteroaryl, —NR⁴⁶C(O)O-substituted heteroaryl, —NR⁴⁶C(O)O-heterocyclic, and —NR⁴⁶C(O)O-substituted heterocyclic where R⁴⁶ is hydrogen or alkyl, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “oxythiocarbonylamino” refers to the groups —NR⁴⁶C(S)O-alkyl, —NR⁴⁶C(S)O-substituted alkyl, —NR⁴⁶C(S)O-alkenyl, —NR⁴⁶C(S)O-substituted alkenyl, —NR⁴⁶C(S)O-alkynyl, —NR⁴⁶C(S)O-substituted alkynyl, —NR⁴⁶C(S)O-cycloalkyl, —NR⁴⁶C(S)O-substituted cycloalkyl, —NR⁴⁶C(S)O-aryl, —NR⁴⁶C(S)O-substituted aryl, —NR⁴⁶C(S)O-heteroaryl, —NR⁴⁶C(S)O-substituted heteroaryl, —NR⁴⁶C(S)O-heterocyclic, and —NR⁴⁶C(S)O-substituted heterocyclic where R⁴⁶ is hydrogen or alkyl, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “aminocarbonyloxy” or as a prefix “carbamoyloxy” or “substituted carbamoyloxy” refers to the groups —OC(O)NR⁴⁷R⁴⁷ where each R⁴⁷ is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic; or where each R⁴⁷ is joined to form, together with the nitrogen atom, a heterocyclic or substituted heterocyclic, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “aminocarbonylamino” refers to the group —NR⁴⁹C(O)NR⁴⁹ where each R⁴⁹ is independently selected from the group consisting of hydrogen and alkyl.

The term “aminothiocarbonylamino” refers to the group —NR⁴⁹C(S)NR⁴⁹R⁴⁹ where each R⁴⁹ is independently selected from the group consisting of hydrogen and alkyl.

“Amidino” refers to the group —C(═NR⁵²)NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aryl” or “Ar” refers to a monovalent aromatic carbocyclic group of from 6 to 14 carbon atoms having a single ring (e.g. phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, benzo[1,3]-dioxol-5-yl, 2,3-dihydro-benzo[1,4]dioxin-6-yl, 2,3-dihydro-benzofuran-5-yl, dibenzofuran-4-yl, and the like) provided that the point of attachment is the aryl group. Preferred aryls include phenyl and naphthyl.

The term “substituted aryl” refers to aryl groups, as defined herein, which are substituted with from 1 to 4, particularly 1 to 3, substituents selected from the group consisting of hydroxyl, acyl, acylamino, acyloxy, alkyl, substituted alkyl, alkoxy, substituted alkoxy, alkenyl, substituted alkenyl, alkynyl, substituted alkynyl, amidino, amino, substituted amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl esters, cyano, thiol, alkylthio, substituted alkylthio, arylthio, substituted arylthio, heteroarylthio, substituted heteroarylthio, cycloalkylthio, substituted cycloalkylthio, heterocyclicthio, substituted heterocyclicthio, cycloalkyl, substituted cycloalkyl, guanidino, halo, nitro, heteroaryl, substituted heteroaryl, heterocyclic, substituted heterocyclic, oxycarbonylamino, oxythiocarbonylamino, —S(O)₂-alkyl, —S(Oh-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, and —OSO₂—NR⁵¹R⁵¹, —NR⁵¹S(O)₂—NR⁵¹-alkyl, —NR⁵¹S(O)₂—NR⁵¹-substituted alkyl, —NR⁵¹S(O)₂—NR⁵¹-aryl, —NR⁵¹S(O)₂—NR⁵¹-substituted aryl, —NR⁵¹S(O)₂—NR⁵¹-heteroaryl, —NR⁵¹S(O)₂—NR⁵¹-substituted heteroaryl, —NR⁵¹S(O)₂—NR⁵¹-heterocyclic, —NR⁵¹S(O)₂—NR⁵¹-substituted heterocyclic, where each R⁵¹ is independently selected from hydrogen or alkyl, wherein each of the terms is as defined herein.

The term “aryloxy” refers to the group aryl-O— that includes, by way of example, phenoxy, naphthoxy, and the like.

The term “substituted aryloxy” refers to substituted aryl-O— groups.

The term “aryloxyaryl” refers to the group -aryl-O-aryl.

The term “substituted aryloxyaryl” refers to aryloxyaryl groups substituted with from 1 to 3 substituents on either or both aryl rings as defined above for substituted aryl.

The term “carboxyl” refers to —COOH or salts thereof.

The term “carboxyl esters” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic.

The term “cycloalkyl” refers to cyclic alkyl groups of from 3 to 10 carbon atoms having single or multiple cyclic rings including, by way of example, adamantyl, cyclopropyl, cyclobutyl, cyclopentyl, cyclooctyl, and the like.

The term “substituted cycloalkyl” refers to a cycloalkyl group, having from 1 to 5 substituents selected from the group consisting of oxo (═O), thioxo (═S), alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

The term “cycloalkenyl” refers to cyclic alkenyl (but not aromatic) groups of from 5 to 10 carbon atoms having single or multiple cyclic rings and having at least one site of vinyl (>C═C<) unsaturation within the ring cyclo including, by way of example, cyclopentenyl, cyclooctenyl, and the like.

The term “substituted cycloalkenyl” refers to a cycloalkenyl group, having from 1 to 5 substituents selected from the group consisting of oxo (═O), thioxo (═S), alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic. It is understood that any hydroxyl substitution is not pendent to a vinyl carbon atom.

The term “cycloalkoxy” refers to —O-cycloalkyl groups.

The term “substituted cycloalkoxy” refers to —O-substituted cycloalkyl groups.

“Guanidino” refers to the group —NHC(═NH)NH₂.

The term “halo” or “halogen” refers to fluoro, chloro, bromo, and iodo, and preferably is fluoro, chloro or bromo.

The term “heteroaryl” refers to an aromatic group of from 1 to 15 carbon atoms, preferably from 1 to 10 carbon atoms, and 1 to 4 heteroatoms selected from the group consisting of oxygen, nitrogen, and sulfur within the ring. Such heteroaryl groups can have a single ring (e.g., pyridinyl, furyl, or thienyl) or multiple condensed rings (e.g., indolizinyl or benzothienyl). The nitrogen and/or sulfur ring atoms can optionally be oxidized to provide for the N-oxide or the sulfoxide, and sulfone derivatives. Preferred heteroaryls include pyridinyl, pyrrolyl, indolyl, thiophenyl, thienyl, and furyl.

The term “substituted heteroaryl” refers to heteroaryl groups that are substituted with from 1 to 3 substituents selected from the same group of substituents defined for substituted aryl.

The term “heteroaryloxy” refers to the group —O-heteroaryl, and “substituted heteroaryloxy” refers to the group —O-substituted heteroaryl.

The term “heterocyclyl” or “heterocyclic” refers to a saturated or unsaturated (but not aromatic) group having a single ring or multiple condensed rings, from 1 to 10 carbon atoms, and from 1 to 4 hetero atoms selected from the group consisting of nitrogen, sulfur or oxygen within the ring wherein, in fused ring systems, one or more of the rings can be aryl or heteroaryl provided that the point of attachment is at the heterocycle. The nitrogen and/or sulfur ring atoms can optionally be oxidized to provide for the N-oxide or the sulfoxide, and sulfone derivatives.

The term “substituted heterocyclyl” or “substituted heterocyclic” refers to heterocycle groups that are substituted with from 1 to 3 of the same substituents as defined for substituted cycloalkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic, and “substituted heterocyclyloxy” refers to the group —O-substituted heterocyclic.

“Thiol” or “mercapto” refers to the group —SH. The term “sulfonyl” refers to the group —SO₂H.

“Alkylsulfanyl”, “alkylthio”, and “thioether” refer to the groups —S-alkyl where alkyl is as defined above.

“Thioxo” refers to the atom (═S).

“Substituted alkylthio” and “substituted alkylsulfanyl” refer to the group —S-substituted alkyl where alkyl is as defined above.

“Cycloalkylthio” or “cycloalkylsulfanyl” refers to the groups —S-cycloalkyl where cycloalkyl is as defined above.

“Substituted cycloalkylthio” refers to the group —S-substituted cycloalkyl where substituted cycloalkyl is as defined above.

“Arylthio” or “arylsulfanyl” refers to the group —S-aryl, and “substituted arylthio” refers to the group —S-substituted aryl where aryl and substituted aryl are as defined above.

“Heteroarylthio” or “heteroarylsulfanyl” refers to the group —S-heteroaryl, and “substituted heteroarylthio” refers to the group —S-substituted heteroaryl where heteroaryl and substituted heteroaryl are as defined above.

“Heterocyclicthio” refers to the group —S-heterocyclic, and “substituted heterocyclicthio” refers to the group —S-substituted heterocyclic where heterocyclic, and substituted heterocyclic are as defined above.

“Oxo” refers to the atom (═O) or (—O⁻).

“Sulfonyl” refers to the divalent group —S(O)₂—.

The term “amino acid” refers to any of the naturally occurring amino acids, as well as synthetic analogs (e.g., D-stereoisomers of the naturally occurring amino acids, such as D-threonine), and derivatives thereof. α-Amino acids comprise a carbon atom to which is bonded an amino group, a carboxyl group, a hydrogen atom, and a distinctive group referred to as a “side chain.” The side chains of naturally occurring amino acids are well known in the art, and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine, and tryptophan), substituted arylalkyl (e.g., as in tyrosine), and heteroarylalkyl (e.g., as in histidine). Unnatural amino acids are also known in the art, as set forth in, for example, Williams, ed. (1989) Synthesis of Optically Active α-Amino Acids, Pergamon Press; Evans et al. (1990) J. Amer. Chem. Soc. 112:4011-4030; Pu et al. (1991) J. Amer. Chem. Soc. 56:1280-1283; Williams et al. (1991) J. Amer. Chem. Soc. 113:9276-9286; and all references cited therein.

The term “pharmaceutically acceptable salt” refers to pharmaceutically acceptable salts of a compound, which salts are derived from a variety of organic, and inorganic counter ions well known in the art, and include, by way of example only, sodium, potassium, calcium, magnesium, ammonium, tetraalkylammonium, and the like, and when the molecule contains a basic functionality, salts of organic or inorganic acids, such as hydrochloride, hydrobromide, tartrate, mesylate, acetate, maleate, oxalate, and the like.

The term “prodrug”, as used herein, refers to compounds of formula I that include chemical groups which, in vivo, can be converted into the carboxylate group on the glycine or alanine substituent of the compounds and/or can be split off from the amide N-atom and/or can be split off from the 4-O atom of the pyrrolo[2,3-c]pyridine, or the 7-O atom of the pyrrolo[3,2-c]pyridine, thiazolo[4,5-c]pyridine, or thiazolo[5,4-c]pyridine; and/or can be split off from the N-atom of the pyridyl ring to provide for the active drug, a pharmaceutically acceptable salt thereof, or a biologically active metabolite thereof. Suitable groups are well known in the art and particularly include: for the carboxylic acid moiety on the glycine or alanine substituent, a prodrug selected from, e.g., esters including, but not limited to, those derived from alkyl alcohols, substituted alkyl alcohols, hydroxy substituted aryls and heteroaryls and the like; amides, particularly amides derived from amines of the formula HNR²⁰R²¹ where R²⁰ and R²¹ are independently hydrogen, alkyl, substituted alkyl, aryl, substituted aryl, and the like; hydroxymethyl, aldehyde and derivatives thereof; and for the pyridyl N atom, a prodrug selected from, e.g., N-oxides and N-alkyl derivatives.

The term “excipient” as used herein means an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent. Binders include, e.g., carbopol, povidone, xanthan gum, etc.; coatings include, e.g., cellulose acetate phthalate, ethylcellulose, gellan gum, maltodextrin, etc.; compression/encapsulation aids include, e.g., calcium carbonate, dextrose, fructose dc, honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams and lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; parenterals include, e.g., mannitol, povidone, etc.; plasticizers include, e.g., dibutyl sebacate, polyvinylacetate phthalate, etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

Similarly, it is understood that the above definitions are not intended to include impermissible substitution patterns (e.g., methyl substituted with 5 fluoro groups or a hydroxyl group attached to an ethenylic or acetylenic carbon atom). Such impermissible substitution patterns are well known to the skilled artisan.

### III. Preparation of the Compounds

The compounds of this disclosure can be prepared with methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991). Rodd's Chemistry of Carbon Compounds, Volumes 1-5, and Supplementals (Elsevier Science Publishers, 1989). Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

### IV. Pharmaceutical Formulations and Routes of Administration

The compositions of the present disclosure can be delivered directly or in pharmaceutical compositions along with suitable carriers or excipients, as is well known in the art. Present methods of treatment can comprise administration of an effective amount of a compound of the disclosure to a subject in need; e.g., a subject having or at risk for anemia due to, e.g., chronic renal failure, diabetes, cancer, AIDS, radiation therapy, chemotherapy, kidney dialysis, or surgery. In a preferred embodiment, the subject is a mammalian subject, and in a most preferred embodiment, the subject is a human subject.

An effective amount of such agents can readily be determined by routine experimentation, as can the most effective and convenient route of administration, and the most appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the disclosure may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the disclosure to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.)

Pharmaceutical dosage forms of a compound of the present disclosure may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present disclosure can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present disclosure can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the disclosure. Transdermal or topical administration would be preferred, for example, in situations in which local delivery with minimal systemic exposure is desired.

For administration by inhalation, or administration to the nose, the compounds for use according to the present disclosure are conveniently delivered in the form of a solution, suspension, emulsion, or semisolid aerosol from pressurized packs, or a nebuliser, usually with the use of a propellant, e.g., halogenated carbons derived from methan and ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the disclosure, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the disclosure are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the disclosure and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the disclosure, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD500 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compositions comprising a compound of the disclosure formulated in a compatible pharmaceutical carrier may also be prepared, placed in an appropriate container, and labeled for treatment of an indicated condition.

These and other embodiments of the present disclosure will readily occur to those of ordinary skill in the art in view of the disclosure herein and are specifically contemplated.

### EXAMPLES

The disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Methods

Eleven lumbar spines (T12/L1-L5/S1) were collected fresh frozen cadavers consisting of 5 males and 6 females with a mean age of 82.5 years ±10.2. The cross-sections of the lumbar disc were visually categorized into Thompson grades 1 to 5 and corroborated with fluoroscopic imaging. The grade 1 (“Control”) and grade 5 (“Degenerate”) discs were used (total 27 discs). The discs were separated into annulus fibrosus (AF) and nucleus pulposus (NP) tissues. The Control discs were tested mechanically and then divided to undergo 0, 2, 4, 8, and 10 days of ribosylation in 0.6M ribose at 37° C. The treated Control discs, along with the degenerated discs were then tested mechanically using a dynamic microindentation device in 0.15M PBS at 20° C. using the Biopent 1000 reference point indentation system (Active Life Scientific, CA). A 1.47 mm diameter cylindrical probe cyclically indented the tissue to a depth of 300 μm at 1 Hz to generate a force displacement curve. Three distinct sites per AF/NP were measured and averaged between the sites. The resulting force-deformation curves, accounting for the probe geometry, were used to compute elastic modulus and tan δ.

Following mechanical measurements, the O-day and 4-day treated Control NP tissues were incubated in 0.15M of a thiazolium salt known to cleave AGEs products (Oturai P S, Christensen M, Rolin B, Pedersen K E, Mortensen S B, Boel E. Effects of advanced glycation end-product inhibition and cross-link breakage in diabetic rats. Metab Clin Exp 2000:49:996-1000.) for 4 days. The degenerated NP tissues were also incubated for 4 days in the thiazolium salt solution, for example, a 0.15M PTB solution. Following the second incubation, all tissues were mechanically assessed again, and then subjected to acid hydrolysis by 6N hydrochloric acid (16 hours, 110C). The collagen crosslinking by AGEs, a sugar-derived posttranslational modification of amino acids, was quantified by measuring the autofluorescence of the acid hydrolysates at 370 nm emission and 440 nm excitation and divided by collagen content (Monnier V M, Kohn R R, Cerami A. Accelerated age-related browning of human collagen in diabetes mellitus. Proc Natl Acad Sci USA 1984; 81:583-587). The collagen content was measured from the acid-hydrolysates using a chloramine T absorbance assay that measured the hydroxyproline, an amino acid on the collagen protein that constitutes approximately 14% of collagen by mass (WOESSNER J F. The determination of hydroxyproline in tissue and protein samples containing small proportions of this imino acid. Arch Biochem Biophys 1961; 93:440-447). T-tests or Analysis of variance was used to determine the differences in treatment groups, and Pearson's correlations were used to determine the correlative relationships between measurements.

Primary spine disc cells were isolated from both human and bovine spine discs. The tissues were digested using a cocktail containing collagenase P, trypsin, and other enzymes to release the primary cells embedded within the tissues (Neidlinger-Wilke C, Liedert A, Wuertz K, Buser Z, Rinkler C, Käfer W, et al. Mechanical stimulation alters pleiotrophin and aggrecan expression by human intervertebral disc cells and influences their capacity to stimulate endothelial migration. Spine 2009; 34:663-669). These cells were then conditioned and allowed to expand for 3 passages. After conditioning in normal cell culture media, the cells were exposed to several conditions: Control, bovine serum albumin, and AGEs. The treatments were dissolved directly in the cell culture media and then the cells were subjected to these conditions for 24 hours. Messenger ribose nucleic acids (mRNA) were then extracted from these cells and amplified into respective complimentary deoxyribose nucleic acids (cDNA). The cDNA sequences were tested for the normalized expression (normalized to the housekeeping gene, GAP-DH) of Interleukin (IL)-4, IL-6 (both of which initiate the inflammatory pain response), and the Receptor-for Advanced Glycation End-products (r-AGEs). The normalized expressions compared to the controls were computed by the delta-delta CT method.

### Results

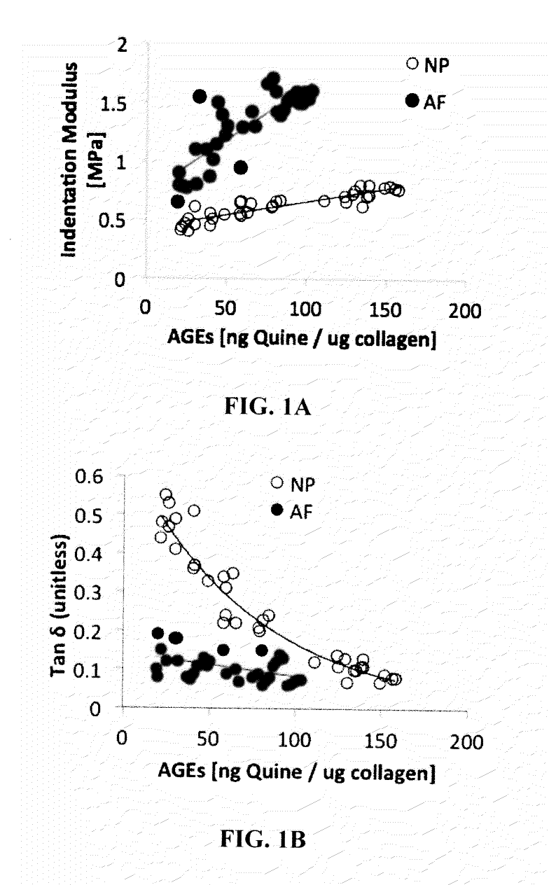

The in vitro ribosylation of the Control AF and NP tissues resulted in a significant increase in the advanced glycation end-products (AGEs). Correspondingly, the changes in AGEs resulted in changes in the elastic and viscoelastic behavior of the tissues. FIG. 1A shows the elastic changes corresponding to the increases of AGEs in annulus fibrosus (AF) and nucleus pulposus (NP) tissues. FIG. 1B shows the viscoelastic changes corresponding to the increases of AGEs in annulus fibrosus (AF) and nucleus pulposus (NP) tissues. As shown in FIG. 1A and FIG. 1B, inducing AGEs in IVD tissues has a significant and differential effect on elastic and viscoelastic properties. The AF indentation modulus was more susceptible to increases in AGEs, while the NP tan δ was more susceptible to AGEs. The elastic behavior of the AF tissue was more affected by the increase of AGEs, while the NP viscoelastic behavior sustained greater changes compared to AGEs. These AGEs-mediated changes are consistent with the changes observed with degeneration.

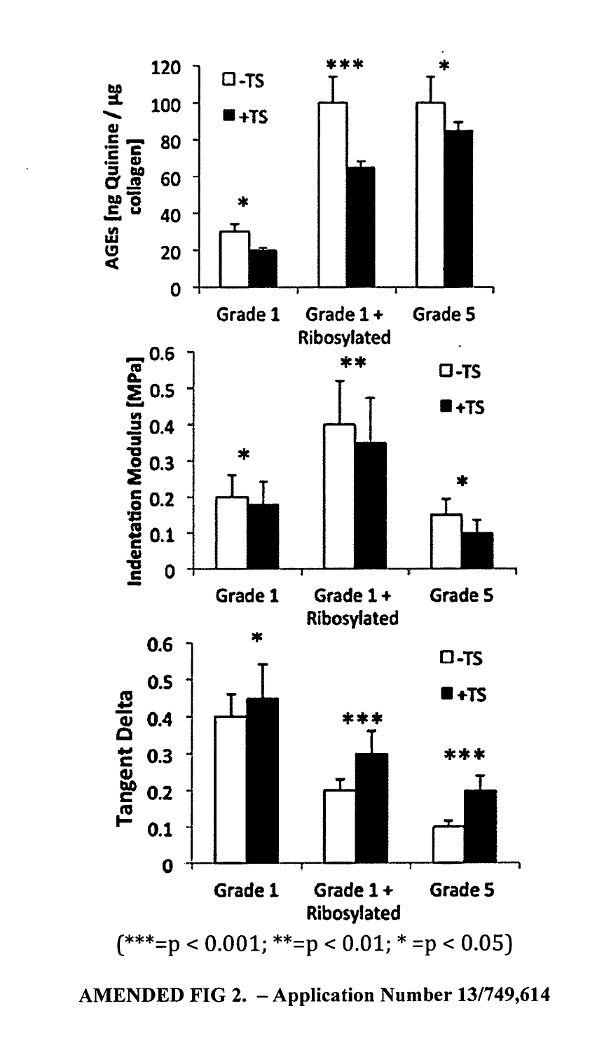

FIG. 2 shows the rescuing effect of cleavage of the advanced glycation end-products using a solution containing N-phenacyl thiazolium bromide in the nucleus pulposus (NP) tissues. Treatment by PTB salts resulted in significant reductions in AGEs in the Grade 1, Grade I+Ribosylation, and Grade 5 NP disc tissues. As shown in FIG. 2, cleavage of the advanced glycation end-products using a thazolium salt solution in the NP tissues can affect the mechanical changes mediated by the in vitro ribosylation. Moreover, administration of the thiazolium salts can rescue the changes that natively present in the degenerated discs by reducing the tissue concentration of AGEs and restoring the mechanical properties closer to the Control (healthy state).

Correspondingly, these changes in AGEs also resulted in changes in both the indentation modulus and tan δ of the thiazolium-treated tissues.

Research in the etiology of pain has been able to show that nerve root compression typically leads to an increase in inflammatory cytokines in the area of compression. Decreasing the inflammation can help alleviate the pain. Applicants find that modulating the expression of the Receptor for Advanced Glycation Endproducts (RAGE) can directly up and down regulate inflammatory cytokines associated with pain. The invention uses AGE breakers to reduce the activation of RAGE, other ways include the inhibition of RAGE using antibodies (e.g. eS-RAGE) to manipulate the downstream response of RAGE. This therapeutic agent will be delivered via an epidural injection directly to the area of nerve root compression, as a means to decrease inflammation and pain, or directly into the diseased disc to locally improve mechanical properties, rehydrate the disc, and suppress the secretion of inflammatory cytokines related to pain. Furthermore, such a therapeutic would have regenerative effects on the intervertebral cells, thus gradually guiding the regeneration of the disc and guide the disc to a healthy status.

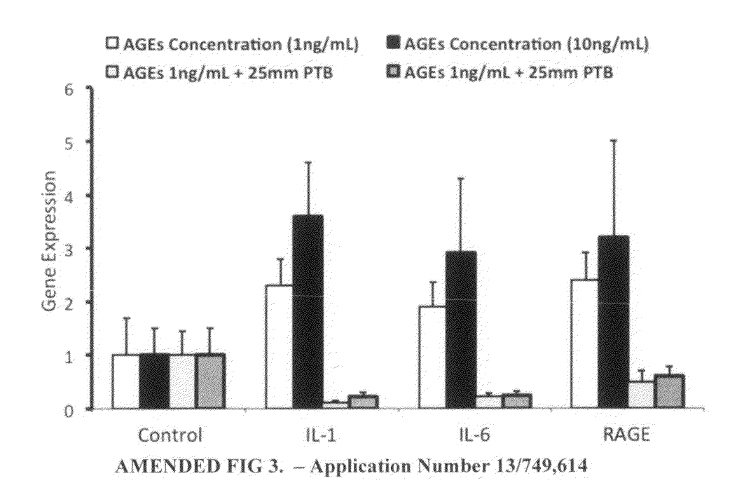

FIG. 3 shows the effect of N-phenacyl thiazolium bromide administration in reducing inflammatory cytokines in primary spinal disc cells. Interleukin-1 and -6 expression was observed to decrease with PTB administration. These effects are confirmed by the reduction in the expression of the Receptor-for-Advanced Glycation End-Products.

The cellular inflammatory response, marked by the cytokines IL-4 and IL-6, were significantly increased by the presence of AGEs within the cell culture media and this response occurs in a dose-dependent manner. The concurrent administration of PTB along with AGEs suppresses this response (FIG. 3), demonstrating that PTB can reverse the painful effects on cells mediated by AGEs.

Although disc degeneration is coupled with changes in cell behavior, matrix composition, and changes in tissue-level mechanics of the IVD, the results show here that modulating the levels of the AGEs can produce specific changes in elastic and viscoelastic properties differentially in the AF and NP tissues that are similar to that of degeneration.

Furthermore, reduction in the level of AGEs restores the tissue functional mechanical behavior comparable to a lesser degree of degeneration.

Thus, the invention includes a pharmaceutical compound for the curative or prophylactic treatment of degenerative disc disease in a human being or animal, or a pharmaceutically acceptable salt thereof, together with a pharmaceutically acceptable diluent or carrier.

The invention also provides a method of treating a human being, or an animal, to cure or prevent degenerative disc disease, which comprises treating said human being or animal with an effective amount of a compound, or a pharmaceutically acceptable salt thereof, or a pharmaceutical composition containing either entity.

Moreover, the invention includes the use of an AGE breaker and an AGE inhibitor, or a pharmaceutically acceptable salt thereof, or a pharmaceutical composition containing either entity, for the manufacture of a medicament for the curative or prophylactic treatment of degenerative disc disease in a human being or animal.

The contents of the articles, patents, and patent applications, and all other documents and electronically available information mentioned or cited herein, are hereby incorporated by reference in their entirety to the same extent as if each individual publication was specifically and individually indicated to be incorporated by reference.

Applicants reserve the right to physically incorporate into this application any and all materials and information from any such articles, patents, patent applications, or other physical and electronic documents.

The disclosures illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claims. Thus, it should be understood that although the present disclosure has been specifically disclosed by preferred embodiments and optional features, modification and variation of the disclosures embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein. Other embodiments are within the following claims. In addition, where features or aspects of the disclosure are described in terms of Markush groups, those skilled in the art will recognize that the disclosure is also thereby described in terms of any individual member or subgroup of members of the Markush group.

1. A method for treating or preventing a degenerative disc disease in a patient in need thereof, comprising administering to the patient a therapeutically effective amount of an advanced glycation endproduct (AGE) breaker or inhibitors.

2. The method of claim 1, wherein the degenerative disc disease comprises an intervertebral disc (IVD) degeneration disease.

3. The method of claim 1, wherein the AGE breaker or inhibitors is a compound selected from the group consisting of a ureido, an aryl carboxamido phenoxyisobutyric acids and an N-phenacyl-thiazolium, a pharmaceutically acceptable salt thereof, or a biologically active metabolite thereof.

4. The method of claim 1, wherein the AGE breaker is a compound having Formula I: 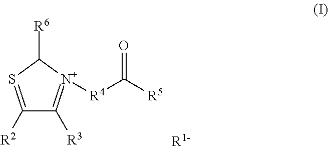
wherein:
R¹ is halo;
R² and R³ are each independently selected from H, alkyl or substituted alkyl;
R⁴ is alkyl or substituted alkyl;
R⁵ is phenyl or substituted phenyl; and
R⁶ is H, alkyl or substituted alkyl.

5. The method of claim 4, wherein R⁵ is phenyl or phenyl substituted with one or more halo, hydroxyl, or C₁-C₅ alkyl.

6. The method of claim 5, wherein R⁵ is phenyl.

7. The method of claim 4, wherein R⁴ is C₁-C₅ alkyl.

8. The method of claim 4, wherein R⁶ is H or methyl.

9. The method of claim 4, wherein the AGE breaker is a compound having Formula II: 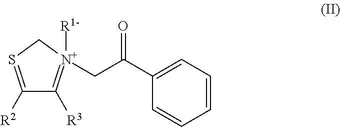
wherein:
R¹ is halo; and
R² and R³ are each independently selected from H, alkyl or substituted alkyl.

10. The method of claim 9, wherein R² and R³ are each independently selected from H or C₁-C₁₀ alkyl.

11. The method of claim 4, wherein the compound is selected from the group consisting of:
N-phenacyl thiazolium bromide (N-PTB);
N-phenacyl-4,5-dimethylthiazolium bromide (PMTB);
dimethyl-3-phenacylthiazolium chloride;
L-bis-[4-(4-chorobenzamidophenoxy isobutryl) cystin (LR20);
4-(3,5-dichlorophenylureido)-phenoxyisobutyryl-1-amidocylohexane-1-carboxylic acid (LR23);
methylene bis[4,4′-(2-chlorophenylureidophenoxyisobutyric acid)](LR90);
1,1-dimethylbiguanide (metformin); and
5-aminosalicylic acid (5-ASA).

12. The method of claim 1, wherein the AGE breaker is administered orally, through intravenous injection, or through direct delivery into an afflicted site.

13. A method for inhibiting the progression of a degenerative disc disease in a patient in need thereof, comprising administering to the patient a therapeutically effective amount of an advanced glycation endproduct (AGE) breaker.

14. A method for reducing the amount of an advanced glycation endproduct (AGE) in an intervertebral disc (IVD) in a patient that suffers from an IVD degeneration disease, comprising administering to the patient a therapeutically effective amount of an advanced glycation endproduct (AGE) breaker or inhibitor.
